(19) United States
(12) Patent Application Publication
(10) Pub. No.: US 2025/0049688 A1
(43) Pub. Date: Feb. 13, 2025

(54) COMPOSITION FOR COSMETIC USE

(71) Applicant: PROFESSIONAL DERMA SA, Lugano (CH)

(72) Inventors: Maurizio RAGNI, Milano (IT); Edoardo CONTI, Lugano-Paradiso (CH)

(73) Assignee: PROFESSIONAL DERMA SA, Lugano (CH)

(21) Appl. No.: 18/691,053

(22) PCT Filed: Sep. 15, 2022
(86) PCT No.: PCT/IB2022/058719

§ 371 Date: Mar. 12, 2024

(30) Foreign Application Priority Data
Sep. 16, 2021 (IT) 102021000023903

(51) Int. Cl. A61K 8/73; A61K 8/44; A61K 8/49; A61K 8/64; A61Q 19/08

(52) U.S. Cl. CPC A61K 8/735; A61K 8/44; A61K 8/4913; A61K 8/64; A61Q 19/08; A61K 2800/91

(57) ABSTRACT

An injectable composition includes a specific combination of low and high molecular weight, non cross-linked hyaluronic acid, in combination with a specific amino acid composition. In a cosmetic use of the composition, which when injected into dermis, the composition is able to counteract the decrease in proteins of the Extra Cellular Matrix and skin hydration due to ageing, especially induced by oxidative stress.

### CROSS-REFERENCE TO RELATED APPLICATIONS

This application is a § 371 U.S. National Phase of International Patent Application No. PCTIB2022/058719, filed Sep. 15, 2022, which claims priority of Italian Patent Application No. 102021000023903, filed Sep. 16 2021, the entire contents of all of which are incorporated by reference herein as if fully set forth.

### TECHNICAL FIELD

The present invention relates to injectable compositions comprising specific combinations of low and high molecular weight hyaluronic acid (HA), non cross-linked, in combination with a specific composition of amino acids (AA) capable of counteracting the decrease of proteins of the Extra Cellular Matrix (ECM) and skin hydration due to ageing induced by oxidative stress.

### BACKGROUND

In humans, the residence time of the injected hyaluronic acid in its natural state is a few days, since the polymer chains are easily degraded by the enzymes and free radicals present in the body and to overcome this problem most of the available compositions on the market are prepared using cross-linking processes of the hydroxyl groups of HA by means of a chemical cross-linker. The final effect on the tissues of such compositions can be controlled by changing the crosslinking density with various crosslinkers; this approach, however, has an important disadvantage, the cross-linked fillers based on HA are in fact very dense and difficult to inject.

It is therefore an object of the present invention to provide alternative and improved injectable compositions to counteract damage from photoageing and improve collagenogenesis in dermis.

### SUMMARY

It is an object of the present invention an injectable composition comprising a specific combination of low and high molecular weight hyaluronic acid (HA), non cross-linked, in combination with a specific composition of amino acids (AA).

In particular, the composition according to the present invention comprising:
Sodium hyaluronate with a molecular weight of 100-400 kDa non cross-linked, in a concentration between 7 and 20 mg/ml
Non crosslinked sodium hyaluronate with a molecular weight of 2000 kDa or higher, in a concentration between 10 and 25 mg/ml
a mixture of amino acids comprising:
   a. Glycine 6-12.5 mg/ml
   b. L-Proline and/or L-Hydroxyproline 5-8 mg/ml
   c. L-Alanine 1-5 mg/ml
   d. L-Valine 1-5 mg/ml
   e. L-Leucine 1-5 mg/ml
   f. L-Lysine HCl 1-5 mg/ml (hydrochloride)
   g. L-arginine HCl 1-5 mg/ml (hydrochloride)

The skin is the largest organ in the body, with a total surface area of about 2 square meters in adults and performs many fundamental functions, including thermoregulation, defense against pathogens and damage caused by UV rays and waterproof barrier; it is connected to the brain via a wide network of nerves and cells and acts as an environmental sensory apparatus (1, 3). The skin is made up of three layers: the epidermis, the outer layer, comprises different types of cells such as squamous cells, basal cells and melanocytes. The dermis is the intermediate skin layer; in addition to blood and lymphatic vessels, hair follicles and sweat glands, it mainly contains bundles of collagen fibers and fibroblasts. The hypodermis is the deepest layer and contains adipose tissue, hair follicles, sensory neurons and blood vessels (3, 2, 4). In addition to the cellular one, one of the major components of the skin is the extracellular matrix (ECM), a complex composition of macromolecules consisting of water, polysaccharides (glycosaminoglycans such as hyaluronic acid, chondroitin sulphate, dermatan sulfate, heparan sulfate and keratan sulfate) and proteins with collagen, fibronectin, laminin, proteoglycans and elastin, the most abundant (5, 6, 7, 8). ECM is mainly produced by fibroblasts, mesenchymal cells that also play a vital role in tissue development, maintenance and repair. ECM gives tissues their ECM mechanical and peculiar properties, but also plays an important role in the regulation of cellular functions; cell interaction with ECM is mediated by specific receptors and not only promotes cell adhesion and migration, but also regulates cell differentiation and gene expression, as well as playing a pivotal role in wound healing (6, 7). In particular, ECM gives the skin its characteristics of elasticity, resistance and compressibility. The most important protein responsible for skin elasticity is elastin, one of the major structural proteins of the ECM, which comprises about 2% of the total proteins of dermis; from a structural point of view, elastin alternates the characteristics of the hydrophobic domain and the hydrophilic domain; it is often organized in repeated short units of three to nine amino acids enriched with glycine, proline, alanine, leucine and valine.

At the origin of the fundamental elastogenesis process there are fibroblasts, which synthesize and secrete the precursor tropoelastin (ELN), a soluble monomer, into the extracellular space, which then, forming a highly cross-linked insoluble polymer composed of covalently bonded tropoelastin molecules, generates elastin in the ECM (9, 10, 11). The other most widespread structural protein in ECM is collagen, which is also the most present protein in mammals; collagen includes a family of molecules that are different both from a structural and functional point of view, with 28 members, in vertebrates numbered with Roman numerals (I-XXVIII), encoded by 28 different genes, among which type I is the most present in human beings. Despite the high heterogeneity of the various types, all members of the family possess the characteristic triple helix structure consisting of three a chains, which can be formed by identical collagen chains (homotrimers) or by the union of different collagen isoforms (heterotrimers). All collagen isoforms contain repeating domains of the Gly-X-Y tripeptide, where the X and Y position is often occupied by proline and hydroxyproline; these n (Gly-X-Y) repetitions are necessary to join the triple helix structure and, depending on the type of collagen, proline and lysine residues are also useful, as they are important sites of post-translational modifications (12,13).

The structure and function of the skin are characterized by sequential and cumulative alterations and include changes in both the vitality and proliferation of cellular components as well as the expression and production of the extracellular matrix. A reduction in the function of the ECM causes both the loss of elasticity and resilience and the appearance of wrinkles, which is a drawback of skin ageing (14,15). In addition, another main feature of skin ageing is dryness and loss of skin hydration.

Hyaluronic acid (HA) glycosaminoglycan is a key molecule for skin hydration, as it has the unique and fundamental ability to bind and retain water molecules (16); in addition to hydration, HA also plays an important role in wound healing, in the migration of fibroblasts, in the immune response and in the development of tumors (16). The size of HA greatly affects its functions; large molecular HA, typically more than 1,000 kDa, is antiangiogenic and immunosuppressive, while smaller HA polymers are powerful inducers of inflammation and angiogenesis. But above all, ageing not only causes a reduction in the synthesis of HA, but also causes the production of harmful HA molecules of smaller size (16, 17, 18).

We are still far from fully clarifying the multiple causes of ageing; the main ones comprise, among others, glycation, telomeric shortening, secondary reactions, mutations and protein aggregation. However, one of the main and well known factors of organic ageing is the production of reactive oxygen species (ROS); in fact already in 1956 the theory of ageing mitochondrial free radicals was advanced, which is mainly based on the ROS generation as a by-product of mitochondrial breathing (19,20). Due to continuous exposure to UV rays, the production of ROS in this organ, in addition to an endogenous (intrinsic) mitochondrial origin, is also due to an exogenous (extrinsic) source; this makes the amount of free radicals in the skin particularly large, triggering the skin photoageing process (21,22). One of the main consequences of skin exposure to UV rays is the accelerated turnover of ECM, which is a normal and fundamental process for maintaining healthy tissues, during which aged proteins are degraded and replaced with newly synthesized proteins (14,23). However, UV irradiation generates intracellular ROS, such as superoxide anion (O₂⁻) and hydrogen peroxide (H₂O₂), resulting in the synthesis of matrix metalloproteinase (MMP) (24,25). Thus the digestion of the ECM component by the MMP, without a simultaneous increase in the production of new ECM proteins, alters the balance in the turnover that exists between synthesis and degradation, to the benefit of the latter. The consequence is a loss of collagen, elastin and fibrillin fibers, with a consequent reduction in skin elasticity and the appearance of wrinkles (21,22).

Another important effect of oxidative stress is the increase in the percentage of cellular senescence, which also plays a fundamental role in skin ageing; senescent fibroblasts no longer split and acquire a secretory phenotype associated with senescence (SASP) (26), with increased secretion of proinflammatory cytokines and chemokines, the remodeling protease of ECM; moreover, SASP increases the quantity of ROS, thus creating a vicious circle of free radical production that ultimately cause premature ageing (26, 27, 28).

To prevent and/or counteract the harmful effects of oxidative stress on skin photoageing, various therapeutic and nutritional approaches have been proposed; an important strategy is represented by the prevention of remodeling of the ECM induced by oxidative stress by stimulating the synthesis of new ECM proteins, especially collagen and elastin, in order to prevent the loss of skin elasticity (29, 30, 31). In addition, by improving the translation of the ECM protein by stimulating the fibroblasts vitality, senescence induced by oxidative stress is also blocked and therefore the onset of skin ageing is delayed.

It is known that amino acids, the building blocks of proteins, when placed as substrates, stimulate the translation of proteins. However, specific amino acid compositions, only if correctly identified and administered, can promote the synthesis of a particular subset of proteins. To date, there are several compositions used to counteract oxidative stress and skin ageing; these compositions are in particular designed to be administered by injection. Currently, there are numerous compositions available for intradermal injection, with the aim either to create volume (e.g. fillers with cross-linked hyaluronic acid (HA)) or to provide a long-term effect by inducing neocollagenesis.

In a preferred embodiment, the composition according to the invention comprises:
9 mg/ml of Glycine
6.5 mg/ml of Proline
2 mg/ml of Alanine
2.5 mg/ml of Valine
1 mg/ml of Leucine
2.5 mg/ml of Lysine HCl
1.5 mg/ml of Arginine HCl

In one embodiment the composition according to the invention comprises sodium hyaluronate with a molecular weight of 100-400 kDa that is present in a 16 mg/ml concentration and sodium hyaluronate with a molecular weight of at least 2000 kDa that is present in a 16 mg/ml concentration.

In one embodiment, sodium hyaluronate with a molecular weight of 100-400 kDa is present in a 12 mg/ml concentration and sodium hyaluronate with a molecular weight of 2000 kDa is present in a 20 mg/ml concentration.

In a preferred embodiment, the composition according to the invention comprises a total sodium hyaluronate concentration per ml greater than 25 mg/ml.

In a preferred embodiment, the composition has a pH preferably between 6.8 and 7.5, even more preferably between 7 and 7.3.

In a preferred embodiment, the composition according to the invention comprises at least one of pharmaceutically acceptable excipients or adjuvants, a buffer, preferably phosphate buffer, an anesthetic agent, preferably a local anesthetic agent.

In a preferred embodiment, the composition according to the invention comprises at least one biomimetic peptide selected from Acetyldecapeptide 3 of SEQ ID NO: 1, Oligopeptide 24 of SEQ ID NO: 2, Acetyltetrapeptide 5 of SEQ ID NO: 3, Vialox Pentapeptide-3 of SEQ ID NO: 4, Acetyl Hexapeptide 8 of SEQ ID NO: 5, Myristoyl Pentapeptide-8 of SEQ ID NO: 6, peptide GHK-Cu of sequence Gly-His-Lys-Cu, Tripeptide-29 of sequence H-Gly-Pro-Hyp-OH, Octapeptide-3 of SEQ ID NO: 7, MATRIXYL of SEQ ID NO: 8, Hexapeptide of SEQ ID NO: 9.

The present invention also relates to a kit comprising an injectable composition as described above, preferably in the form of a gel, comprised in a pre-filled syringe and optionally comprising the instructions for use.

The present invention also relates to the use of the compositions as described above in the treatment of skin deterioration and/or senescence, elastosis and dermoepidermal atrophy or other pathologies caused by oxidative stress and for cosmetic applications, preferably for the treatment of photoageing, skin depressions, scars, imperfections and asymmetries of the face, of wrinkles and skin lines preferably of the face more preferably glabellar wrinkles, nasolabial folds, folds of the chin, marionette wrinkles, buccal wrinkles, peri-oral wrinkles, crow's feet.

Finally, it is an object of the present invention the use of the composition or kit according to the invention to stimulate the collagen synthesis and a non-therapeutic method of skin treatment of a subject, comprising the intradermal injection of the composition.

Further objects will become apparent from the following detailed description.

### BRIEF DESCRIPTION OF THE DRAWINGS

FIG. 1—Catalase (CAT) and Ink4 mRNA expression in BJ fibroblasts pretreated with the three compositions (AA1, AA2 or AA3) or without pretreatment (culture medium only) (CT) and then treated with hydrogen peroxide (H₂O₂)) or not treated at all (NT).

FIG. 2—mRNA expression of tropoelastin (ELN) fibrillin (FBN) and collagen isoform IV (Col4a1) in BJ fibroblasts pretreated with the three compositions (AA1, AA2 or AA3) or without pretreatment (culture medium only) (CT) and then treated with hydrogen peroxide (H₂O₂) or untreated at all (NT).

FIG. 3—Analysis of the proliferation, by MTT colorimetric assay, of BJ fibroblasts before treatment (0 hours) and after 24, 48 and 72 hours of incubation with the two mixtures AA4 and AA3 at different final concentrations (0.1%, 0.3%, 0.5%).

FIG. 4—Gene expression of the mRNA of two collagen isoforms (col1a1 and col4a1) after 72 hours of incubation in BJ fibroblasts pretreated with the 2 compositions (AA4, or AA3).

The values 0.1%, 0.3%, 0.5% refer to different final concentrations of the two mixtures.

### DETAILED DESCRIPTION OF THE PREFERRED EMBODIMENTS

A new composition was therefore developed by the inventors to counteract the skin ageing process and stimulate collagenogenesis. This composition comprises a specific combination of medium and high molecular weight hyaluronic acid (HA), non cross-linked, in combination with a specific composition of amino acids (AA) in order to counteract the decrease in ECM proteins and skin hydration due to ageing induced by oxidative stress.

In one embodiment, the composition according to the invention also further comprises biomimetic peptides selected for their effect.

Therefore, the subject of the present invention is a new composition, comprising a composition of amino acids and hyaluronic acid at different molecular weights, non cross-linked, able both to stimulate the synthesis of elastin and collagen, and to perform a protective function against ROS and senescence induced by oxidative stress.

The present invention also relates to a composition further comprising biomimetic peptides.

The composition according to the invention (AA3) is composed as follows:
non cross-linked hyaluronic acid having a 100-400 kDa molecular weight
non cross-linked hyaluronic acid having a 2000 kDa or higher molecular weight
25 mg/ml total of amino acids as follows:
   i. Glycine 6-12.5 mg/ml
   ii. L-Proline and/or L-Hydroxyproline 5-8 mg/ml
   iii. L-Alanine 1-5 mg/ml
   iv. L-Valine 1-5 mg/ml
   v. L-Leucine 1-5 mg/ml
   vi. L-Lysine HCl (hydrochloride) 1-5 mg/ml
   vii. L-arginine HCl (hydrochloride) 1-5 mg/ml.

In a preferred embodiment the composition comprises
i. 9 mg/ml of Glycine
ii. 6.5 mg/ml of Proline
iii. 2 mg/ml of Alanine
iv. 2.5 mg/ml of Valine
v. 1 mg/ml of Leucine
vi. 2.5 mg/ml of Lysine HCl
vii. 1.5 mg/ml of Arginine HCl

In a preferred embodiment, the composition is that shown in table 1.

TABLE 1

| Composition AA3 | mg/ml (MW) |
|---|---|
| Sodium hyaluronate | 7-20(100-400) |
| | 10-25 (≥2000) |
| Glycine | 9 |
| Proline | 6.5 |
| Alanine | 2 |
| Valine | 2.5 |
| Leucine | 1 |
| Lysine | 2.5 |
| Arginine | 1.5 |
| Total AA | 25 |

The composition according to the invention optionally comprises a concentration comprised between 0.005 mg/ml and 0.080 mg/ml preferably between 0.005 mg/ml and 0.05 mg/ml more preferably between 0.005 mg/ml and 0.02 mg/ml of at least one among the following peptides:
Acetyldecapeptide 3 of SEQ ID NO: 1 Ac-Tyr-Arg-Ser-Arg-Lys-Tyr-Thr-Ser-Trp-Tyr-NH₂,
Oligopeptide 24 of SEQ ID NO: 2 H-RGDGCMYIEGGGG-OH,
Acetyltetrapeptide 5 of SEQ ID NO: 3-Ac-β-Ala-His-Ser-His-OH,
Vialox Pentapeptide-3 of SEQ ID NO: 4 Gly-Pro-Arg-Pro-Ala-NH₂,
Acetyl Hexapeptide-8 (Argireline) of SEQ ID NO: 5 Ac-Glu-Glu-Met-Gln-Arg-Arg-NH₂,
Myristoyl Pentapeptide-8 of SEQ ID NO: 6 Myr-RGDGK-NH₂,
GHK-Cu peptide of Gly-His-Lys-Cu sequence not reported in the sequence listing (having only 3 amino acids),
Tripeptide-29 (Collagen tripeptide) of sequence H-Gly-Pro-Hyp-OH not reported in the sequence listing (having only 3 amino acids).

The composition according to the present invention can further comprise at least one of:
Octapeptide-3 of SEQ ID NO: 7 Ac-Glu-Glu-Met-Gln-Arg-Arg-Ala-Asp-NH₂,
MATRIXYL of SEQ ID NO: 8 Lys-Thr-Thr-Lys-Ser,
Hexapeptide of SEQ ID NO: 9 Val-Gly-Val-Ala-Pro-Gly,

The concentration indicated above is intended for each peptide.

In a preferred embodiment, the composition according to the invention comprises a total sodium hyaluronate concentration per ml greater than 25 mg/ml.

The composition according to the present invention may further comprise pharmaceutically acceptable excipients or adjuvants.

The composition according to the present invention can further comprise a buffer, for instance a phosphate buffer, to adjust the pH.

The pH of the composition is preferably between 6.8 and 7.5, even more preferably between 7 and 7.3.

The composition according to the present invention can further comprise an anesthetic agent, in particular a local anesthetic, preferably lidocaine, in a concentration between 0.1% and 0.4%, preferably between 0.2% and 0.3%.

In order to verify the improved effect on collagenogenesis of the composition according to the invention in comparison with the compositions currently present on the market, comparative tests were carried out wherein the composition according to the invention was compared with other products currently available on the market and whose compositions are shown in tables 2 and 4: one (AA1) containing 33 mg/ml total of amino acids (as per table 2) plus 10 mg/ml of hyaluronic acid (100 kDa) and one (AA2) containing only 16 mg/ml of cross-linked hyaluronic acid (80-100 kDa) +16 mg/ml of cross-linked hyaluronic acid (1100-1400 kDa). AA4 contains 32 mg/ml of low molecular weight hyaluronic acid (100-400 kDa) and the same mix of amino acids as the composition according to the invention but lacking lysine.

TABLE 2

| | AA1 | AA2 | AA3 |
|---|---|---|---|
| Composition | mg/ml (MW) | mg/ml (MW) | mg/ml (MW) |
| Sodium hyaluronate (MW) | 10 (200) | 16 (80-100) | 16 (100-400) |
| | | 16 (1100-1400) | 16 (2000) |
| Glycine | 10 | | 9 |
| Proline | 8 | | 6.5 |
| Alanine | 7 | | 2 |
| Valine | 5 | | 2.5 |
| Leucine | 2 | | 1 |
| Lysine | 1 | | 2.5 |
| Arginine | | | 1.5 |
| Total AA | 33 | | 25 |

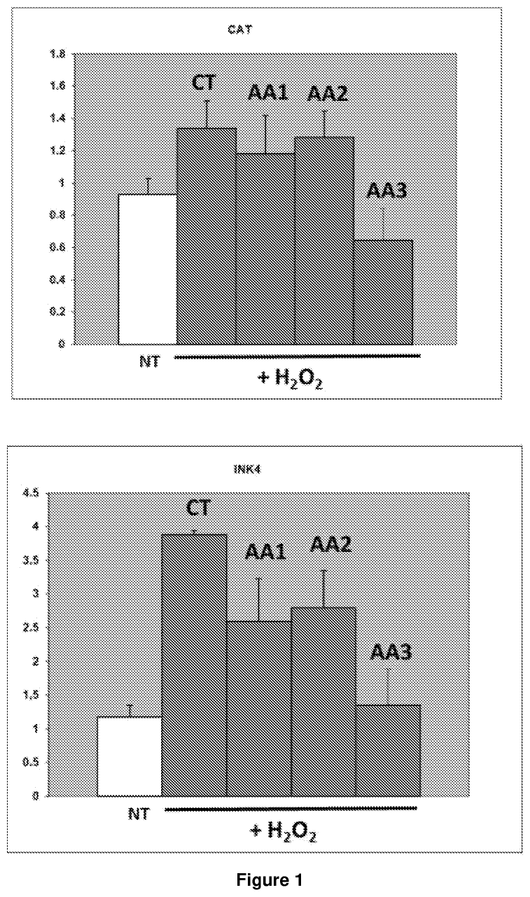

To evaluate the induction of oxidative stress, expression levels of catalase mRNA (CAT), a known ROS scavenger and antioxidant enzyme in a BJ 8 human fibroblast cell line (ATCC CRL-2522) were first analyzed and compared. CAT mRNA levels increase in response to H₂O₂ and therefore constitute a marker of intracellular ROS levels. In FIG. 1 it is possible to observe, as expected, the CAT mRNA induced by the treatment with H₂O₂ (+48% compared to untreated cells-NT). The comparison cells were pretreated with compositions AA1, AA2 and AA3, respectively.

The inventors surprisingly found that pretreatment of BJ fibroblasts with AA3 is able to significantly reduce CAT mRNA to levels similar to those of control cells where oxidative stress had not been induced, indicating the property of the composition to counteract the effect of oxidative stress in cells (−51% compared to cells treated only with H₂O₂-CT). On the contrary, neither the pretreatment with the composition AA1 nor with the composition AA2 substantially changed the levels of CAT in (−8% and −4% with respect to the CT).

The composition according to the invention is therefore, compared to the preparations described in the known art, extremely more effective in counteracting the effects of oxidative stress in fibroblasts.

In order to measure the levels of senescence induced by oxidative stress, the mRNA expression levels of p16 (INK4a) (INK4), a well-known marker of senescence whose mRNA levels are very high in senescent cells (36). Also in this case, INK4 mRNA has increased enormously by treatment with H₂O₂, thus confirming the onset of senescence in BJ fibroblasts due to oxidative stress; the comparison cells were also, in this case, respectively pretreated with the compositions AA1, AA2 and AA3.

Similarly to what has been observed for CAT levels, it is possible to observe a high decrease in the expression levels of INK4 mRNA with an AA3-based pretreatment (−65% compared to CT), revealing a lower senescence in the fibroblasts to which composition AA3 was administered. Although a reduction of INK4 mRNA was also recorded in cells pretreated with composition AA1 (−33% compared to CT) and AA2 (−28% compared to CT), the composition AA3 proved to be extremely more effective. Table 3 below summarizes the data discussed above.

TABLE 3
Percentage reduction of mRNA levels in cells treated with the
indicated compositions and with hydrogen peroxide (H₂O₂) compared
to the control treated with hydrogen peroxide only.

| | AA1 | AA2 | AA3 |
|---|---|---|---|
| CAT mRNA | −8% | −4% | −51% |
| INK4 mRNA | −33% | −28% | −65% |

To verify the effect of the composition according to the invention on the oxidative stress-induced reduction of mRNA levels in ECM proteins, the inventors analyzed the mRNA expression of three ECM proteins, namely elastin (ELN), fibrillin (FBN) and Col4a1 collagen.

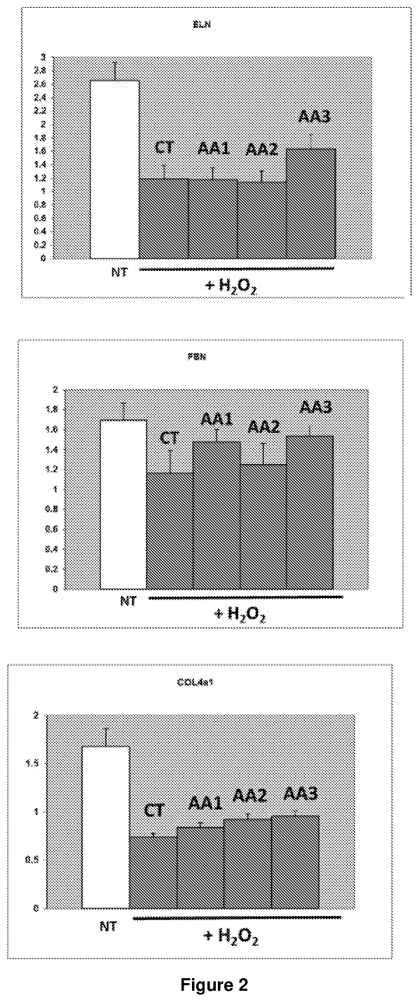

The expression of these mRNAs is strongly influenced by oxidative stress and, as shown in FIG. 2, following a treatment with H₂O₂ the mRNA of all ECM genes undergoes a strong reduction, confirming the deterioration of the ECM due to oxidative stress.

The cells, also in this case, were treated with the 3 compositions reported in table 2; following the treatments, it is possible to observe how the mRNA expression levels of the ELN are not influenced by either the treatment with the composition AA1 or by the treatment with the composition AA2 (−0.9 and −4% compared to the CT).

On the contrary, thanks to the treatment with AA3 it is possible to obtain a restoration (+37% compared to CT) of the expression levels of the ELN mRNA.

Similarly, the mRNA depletion of FBN due to oxidative stress (−31% compared to NT) was almost completely blocked with the composition AA3 (+32% compared to CT) and partially with AA1 (+26% compared to CT), but not with the AA2 (+3%).

Finally, the depletion of col4a1 induced by H₂O₂(−55% compared to NT) improved slightly with the compositions with AA1, AA2 (+13%, +24%) and slightly more significantly with the composition AA3+28% compared to CT.

The composition AA3 not only extremely effectively reduced oxidative stress and senescence of BJ fibroblasts treated with H₂O₂, but also recovered the decrease in ECM mRNA induced by it. In particular, only composition AA3 proved effective against oxidative stress and senescence, but not AA1 or AA2; similarly, only AA3 has restored the expression of ELN. Furthermore, the composition according to the invention compared to the comparative compositions has proved more effective in increasing the mRNA of the FBN and able to restore the levels of Col4a1 together with the other two compositions. ELN is one of the most important proteins of ECM and is responsible for skin elasticity (9,10); also FBN is another fundamental protein for the formation of the elastic fibers of the ECM, whose levels, as is known, decrease with ageing, especially in the case of extrinsic oxidative stress (37). Therefore, the ability of the AA3 composition to restore mRNA levels by counteracting the reduction induced by H₂O₂, demonstrates that this composition acts effectively in counteracting the deterioration of ECM caused by ageing. Among the skin collagen isoforms, type IV (Col4a1) is one of the most affected by ageing; its deterioration also highlights skin senescence and photoageing induced by oxidative stress (38). The composition according to the invention has been shown to be able to partially restore col4a1 mRNA levels decreased following the treatment with H₂O₂.

Of all the compositions tested, the composition according to the invention proved to be the most effective. The greater effectiveness of the composition according to the invention, compared to AA1 and AA2, in counteracting oxidative stress, senescence and the decrease in ECM is due to its particular composition; in fact, although the composition according to the invention contains less total amino acids than AA1, the particular ratios between them and the presence of arginine, in addition to the selected molecular weights of HA, allows to obtain greater beneficial effects on the senescence of fibroblasts and a greater antioxidant power than the comparative compositions.

By way of further comparison, the effect of the composition AA3 according to the invention was tested in comparison with a composition (AA4) lacking both high molecular weight hyaluronic acid and lysine. The compositions are exemplified in table 4.

TABLE 4

| Composition | AA3 (mg/mL) | AA4 (mg/mL) |
|---|---|---|
| Sodium hyaluronate | 16 (100-400 kDa) | 32 (100-400 kDa) |
| | 16 (2000 kDa) | — |
| Glycine | 9 | 9 |
| Proline | 6.5 | 6.5 |
| Alanine | 2 | 2 |
| Valine | 2.5 | 2.5 |
| Leucine | 1 | 1 |
| Lysine | 2.5 | — |
| Arginine | 1.5 | 1.5 |
| Total AA | 25 | 22.5 |

The effects of the compositions were evaluated both by analyzing the proliferation of fibroblasts and on the gene expression of collagen (Col4a1, Cola1a1).

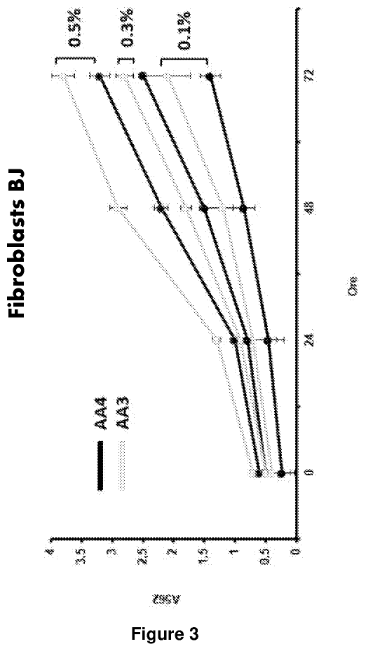

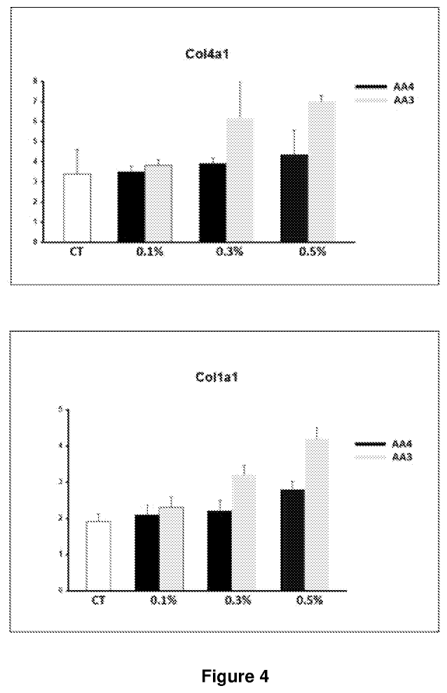

Human BJ fibroblasts were cultured at 70%-80% confluence in F12 medium plus 10% (v/v) fetal bovine serum (FBS) and 2 mM L-glutamine in a 5% CO₂/95% air atmosphere. The cells were then treated with compositions AA3 and AA4 at the final concentrations indicated in FIGS. 3 and 4 (0.1%-0.3%-0.5%). Proliferation was determined using the MTT [3-(4,5-dimethylthiazol-2-yl)-2,5-diphenyltetrazolium bromide] assay. 8×10³ cells/well were seeded in a 96-well plate in 100 μL of medium. The purple formazan crystals were solubilized overnight at 37° C. in 5% SDS/0.1M HCl (100 μL/well) and the absorbance was recorded on a microplate reader at a double wavelength of 570 nm/655 nm at time zero (as a control), 24, 48 and 72 hours. The results are shown in FIG. 3 and show how the composition according to the invention, at each concentration tested, and in a dose-dependent manner, is more effective than the comparative composition in stimulating the proliferation of BJ fibroblasts. Furthermore, the composition according to the invention increases the proliferation of BJ fibroblasts and is more effective than the comparative composition even at the lowest dosage.

Alternatively, at the end of the experimental treatments (72 hours), the cells were used for mRNA extraction. The RNA was isolated using the RNeasy Mini Kit (Qiagen) and the cDNA (1 μg) was synthesized using the iScript (Bio-Rad Laboratories) cDNA synthesis kit. The relative level of gene expression was calculated as 2-ΔΔCT, where ΔΔCT corresponded to the difference between the ΔCT of both treatments and the ΔCT of the untreated group. GAPDH was used as the reference housekeeping gene. The results are shown in FIG. 4 and show how the composition according to the invention is more effective than the comparison composition in promoting the expression of both the analyzed collagen genes; moreover, the composition according to the invention increases the expression of both genes in a dose-dependent manner and at each concentration, proving to be more effective than the comparative composition even at relatively low dosages.

The inventors have therefore developed a composition which, compared to the compositions currently described in the known art, is able to block the harmful effects of oxidative stress, senescence and reduction of ECM proteins in fibroblasts in an extremely more effective way.

According to the present invention, by “injectable” is meant deliverable from syringes under normal conditions at normal pressure and refers to injection into skin, dermis or other tissues to bring the composition to the desired destination site.

The object of the present invention is therefore an injectable composition comprising:
from 7 to 20 mg/ml of non cross-linked sodium hyaluronate having a 100-400 kDa molecular weight,
from 10 to 25 mg/ml of non-cross-linked sodium hyaluronate having a 2000 kDa molecular weight,

A mixture of amino acids comprising:
a. Glycine 6-12.5 mg/ml
b. L-Proline and/or L-Hydroxyproline 5-8 mg/ml
c. L-Alanine 1-5 mg/ml
d. L-Valine 1-5 mg/ml
e. L-Leucine 1-5 mg/ml
f. L-Lysine HCl 1-5 mg/ml
g. L-arginine HCl 1-5 mg/ml

In a preferred embodiment the composition comprises:
a. 9 mg/ml of Glycine
b. 6.5 mg/ml of L-Proline
c. 2 mg/ml of L-Alanine
d. 2.5 mg/ml of L-Valine
e. 1 mg/ml of L-Leucine
f. 2.5 mg/ml of L-Lysine HCl
g. 1.5 mg/ml of L-Arginine HCl

In a preferred embodiment, the composition comprises 16 mg/ml of non-crosslinked sodium hyaluronate with a molecular weight of 100-400 kDa and 16 mg/ml of non-crosslinked sodium hyaluronate with a molecular weight of 2000 kDa or higher.

In another preferred embodiment, the composition according to the invention comprises 12 mg/ml of non-crosslinked sodium hyaluronate with a molecular weight of 100-400 kDa and 20 mg/ml of non-crosslinked sodium hyaluronate with a molecular weight of 2000 kDa.

In a preferred embodiment the composition comprises a total sodium hyaluronate concentration per ml greater than 25 mg/ml.

In one embodiment the composition according to the invention optionally comprises, at a concentration of between 0.005 mg/ml and 0.080 mg/ml preferably between 0.005 mg/ml and 0.05 mg/ml more preferably between 0.005 mg/ml and 0.02 mg/ml at least one of the following peptides:
Acetyldecapeptide 3 of SEQ ID NO: 1 Ac-Tyr-Arg-Ser-Arg-Lys-Tyr-Thr-Ser-Trp-Tyr-NH₂,
Oligopeptide 24 of SEQ ID NO: 2 H-RGDGCMYIEGGGG-OH,
Acetyltetrapeptide 5 of SEQ ID NO: 3 Ac-β-Ala-His-Ser-His-OH,
Vialox Pentapeptide-3 from SEQ ID NO: 4 Gly-Pro-Arg-Pro-Ala-NH₂,
Acetyl Hexapeptide-8 (Argireline) of SEQ ID NO: 5 Ac-Glu-Glu-Met-Gln-Arg-Arg-NH₂,
Myristoyl Pentapeptide-8 of SEQ ID NO: 6 Myr-RGDGK-NH₂,
GHK-Cu peptide of Gly-His-Lys-Cu sequence not reported in the sequence listing (having only 3 amino acids),
Tripeptide-29 (Collagen tripeptide) of sequence H-Gly-Pro-Hyp-OH not reported in the sequence listing (having only 3 amino acids).

The composition according to the present invention can further comprise at least one of:
Octapeptide-3 of SEQ ID NO: 7 Ac-Glu-Glu-Met-Gln-Arg-Arg-Ala-Asp-NH₂,
MATRIXYL of SEQ ID NO: 8 Lys-Thr-Thr-Lys-Ser,
Hexapeptide of SEQ ID NO: 9 Val-Gly-Val-Ala-Pro-Gly,

The composition according to the present invention can further comprise at least one of pharmaceutically acceptable excipients or adjuvants.

The composition according to the present invention may further comprise a buffer, for instance a phosphate buffer, for adjusting the pH. Said pH is preferably between 6.8 and 7.5, even more preferably between 7 and 7.3.

The composition according to the present invention can further comprise an anesthetic agent, in particular a local anesthetic, preferably lidocaine, in a concentration between 0.1% and 0.4%, preferably between 0.2% and 0.3%.

The present invention also relates to a kit comprising an injectable composition according to the invention in the form of a gel in a pre-filled syringe and optionally the instructions for use.

Therefore, an object of the present invention is a composition or a kit as defined above for use in the treatment of skin deterioration and/or senescence, elastosis and dermo-epidermal atrophy caused by oxidative stress.

The composition and the kit according to the invention are also used for the treatment of photoageing, skin depressions, scars, imperfections and asymmetries of the nose, lips, cheeks, perioral region, infraorbital region, facial asymmetries, jaw and chin lines, wrinkles and skin lines for instance of the face by way of non-limiting example glabellar wrinkles, nasolabial folds, folds of the chin, marionette wrinkles, buccal wrinkles, peri-oral wrinkles, crow's feet.

The composition and the kit according to the invention are also used to stimulate the synthesis of collagen and for the cosmetic improvement of soft tissues.

The use according to the present invention is preferably a use in the treatment of a cosmetic condition, however, the composition can also be administered for the treatment of a therapeutic indication.

### EXAMPLES

### Cells and Treatments.

Human BJ fibroblasts were purchased from the American Type Culture Collection (ATCC-CRL-2522) and were grown to a confluence of 70%-80% in an F12 culture medium (ATCC) with the addition of fetal bovine serum at 10% (FBS) (v/v) and 2 mM of L-glutamine and grown in a humidified atmosphere with 5% CO₂/95% air. The cells were pretreated for 24 hours with 1% of the compositions shown in Table 2.

Then, to induce oxidative stress, the cells were treated with 200 μM of H₂O₂ for two hours and a further 48 hours. Untreated cells were plated for control. At the end of the experimental treatments, the cells were used to extract mRNA.

### Total RNA Extraction and Gene Expression Analysis.

RNA was isolated from BJ fibroblasts with the RNeasy Mini Kit (Qiagen), and cDNA (1 μg) was synthesized with the iScript cDNA Synthesis Kit (Bio-Rad Laboratories). A relative level of gene expression of 2-ΔΔCT was calculated, where ΔΔCT corresponds to the difference between the ΔCT of the treated group and the ΔCT of the untreated group. GAPDH was used as a reference for the constitutive gene.

Gene expression levels of CAT, INK4, ELN, COL4a1, FBN were analyzed.

### BIBLIOGRAPHY

1. Kanitakis J. Anatomy, histology and immunohistochemistry of normal human skin. Eur J Dermatol. 2002 July-August; 12(4):390-9; quiz 400-1. PMID: 12095893.
2. Urmacher C. Histology of normal skin. Am J Surg Pathol. 1990 July; 14(7):671-86. doi: 10.1097/00000478-199007000-00008. PMID: 1694059.
3. Yousef H, Alhajj M, Sharma S. Anatomy, Skin (Integument), Epidermis. In: StatPearls
4. Ribeiro, Camila & Leal, Fabiano & Jeunon, Thiago. (2017). Skin Anatomy, Histology, and Physiology. 10.1007/978-3-319-20250-1_1-1.
5. Theocharis A D, Skandalis S S, Gialeli C, Karamanos N K. Extracellular matrix structure. Adv Drug Deliv Rev. 2016 Feb. 1; 97:4-27.
6. Manou D, Caon I, Bouris P, Triantaphyllidou I E, Giaroni C, Passi A, Karamanos N K, Vigetti D, Theocharis A D. The Complex Interplay Between Extracellular Matrix and Cells in Tissues. Methods Mol Biol. 2019; 1952:1-20.
7. Teti A. Regulation of cellular functions by extracellular matrix. J Am Soc Nephrol. 1992 April; 2(10 Suppl): S83-7.
8. Bosman F T, Stamenkovic I. Functional structure and composition of the extracellular matrix. J Pathol. 2003 July; 200(4):423-8.
9. Mithieux S M, Weiss A S. Elastin. Adv Protein Chem. 2005; 70:437-61.
10. Vindin H, Mithieux S M, Weiss A S. Elastin architecture. Matrix Biol. 2019 November; 84:4-16. doi: 10.1016/j.matbio.2019.07.005. Epub 2019 Jul. 10.
11. Schräder CU, Heinz A, Majovsky P, Karaman Mayack B, Brinckmann J, Sippl W, Schmelzer C E H. Elastin is heterogeneously cross-linked. J Biol Chem. 2018 Sep. 28; 293(39):15107-15119
12. Ricard-Blum S. The collagen family. Cold Spring Harb Perspect Biol. 2011 Jan. 1; 3(1)
13. Sorushanova A, Delgado L M, Wu Z, Shologu N, Kshirsagar A, Raghunath R, Mullen A M, Bayon Y, Pandit A, Raghunath M, Zeugolis D I. The Collagen Suprafamily: From Biosynthesis to Advanced Biomaterial Development. Adv Mater. 2019 January; 31(1): e1801651.
14. Birch H L. Extracellular Matrix and Ageing. Subcell Biochem. 2018; 90:169-190.
15. Black L D, Allen P G, Morris S M, Stone P J, Suki B. ECMhanical and failure properties of extracellular matrix sheets as a function of structural protein composition. Biophys J. 2008 Mar. 1; 94(5):1916-29. doi: 10.1529/biophysj.107.107144. Epub 2007 Nov. 9.
16. Dicker K T, Gurski L A, Pradhan-Bhatt S, Witt R L, Farach-Carson M C, Jia X. Hyaluronan: a simple polysaccharide with diverse biological functions. Acta Biomater. 2014 April; 10(4):1558-70.
17. Fallacara A, Baldini E, Manfredini S, Vertuani S. Hyaluronic Acid in the Third Millennium. Polymers (Basel). 2018 Jun. 25; 10(7):701.
18. Papakonstantinou E, Roth M, Karakiulakis G. Hyaluronic acid: A key molecule in skin ageing. Dermatoendocrinol. 2012 Jul. 1; 4(3):253-8. doi: 10.4161/derm.21923. PMID: 23467280; PMCID: PMC3583886.
19. da Costa J P, Vitorino R, Silva G M, Vogel C, Duarte A C, Rocha-Santos T. A synopsis on ageing-Theories, ECMhanisms and future prospects. Ageing Res Rev. 2016 August; 29:90-112.
20. Harman D. Free radical theory of ageing. Mutat Res. 1992 September; 275(3-6):257-66.
21. Fisher G J, Kang S, Varani J, Bata-Csorgo Z, Wan Y, Datta S, Voorhees J J. ECMhanisms of photoageing and chronological skin ageing. Arch Dermatol. 2002 November; 138(11):1462-70. doi: 10.1001/archderm.138.11.1462. PMID: 12437452.
22. Battie C, Jitsukawa S, Bernerd F, Del Bino S, Marionnet C, Verschoore M. New insights in photoageing, UVA induced damage and skin types. Exp Dermatol. 2014 October; 23 Suppl 1:7-12.
23. Kehlet S N, Willumsen N, Armbrecht G, Dietzel R, Brix S, Henriksen K, Karsdal M A. Age-related collagen turnover of the interstitial matrix and basement membrane: Implications of age- and sex-dependent remodeling of the extracellular matrix. PLoS One. 2018 Mar. 29; 13(3): e0194458.
24. Huang H, Du W, Brekken R A. Extracellular Matrix Induction of Intracellular Reactive Oxygen Species. Antioxid Redox Signal. 2017 Oct. 20; 27(12):774-784.
25. Herrmann G, Wlaschek M, Lange T S, Prenzel K, Goerz G, Scharffetter-Kochanek K. UVA irradiation stimulates the synthesis of various matrix-metalloproteinases (MMPs) in cultured human fibroblasts. Exp Dermatol. 1993 March; 2(2):92-7.
26. Campisi J. The role of cellular senescence in skin ageing. J Investig Dermatol Symp Proc. 1998 August; 3(1):1-5.
27. Ghosh K, Capell B C. The Senescence-Associated Secretory Phenotype: Critical Effector in Skin Cancer and Ageing. J Invest Dermatol. 2016 November; 136(11):2133-2139.
28. Russell-Goldman E, Murphy G F. The Pathobiology of Skin Ageing: New Insights into an Old Dilemma. Am J Pathol. 2020 July; 190(7):1356-1369.
29. Lupo M P, Cole A L. CosECMeutical peptides. Dermatol Ther. 2007 September-October; 20(5):343-9
30. Reddy B, Jow T, Hantash B M. Bioactive oligopeptides in dermatology: Part I. Exp Dermatol. 2012 August; 21(8):563-8.
31. Proksch E, Schunck M, Zague V, Segger D, Degwert J, Oesser S. Oral intake of specific bioactive collagen peptides reduces skin wrinkles and increases dermal matrix synthesis. Skin Pharmacol Physiol. 2014; 27(3):113-9.
32. Murakami H, Shimbo K, Inoue Y, Takino Y, Kobayashi H. Importance of amino acid composition to improve skin collagen protein synthesis rates in UV-irradiated mice. Amino Acids. 2012 June; 42(6):2481-9.
33. Murakami H, Shimbo K, Takino Y, Kobayashi H. Combination of BCAAs and glutamine enhances dermal collagen protein synthesis in protein-malnourished rats. Amino Acids. 2013 March; 44(3):969-76.
34. Albaugh V L, Mukherjee K, Barbul A. Proline Precursors and Collagen Synthesis: Biochemical Challenges of Nutrient Supplementation and Wound Healing. J Nutr. 2017 November; 147(11):2011-2017.
35. Nenoi M, Ichimura S, Mita K, Yukawa O, Cartwright I L. Regulation of the catalase gene promoter by Spi, CCAAT-recognizing factors, and a WT1/Egr-related factor in hydrogen peroxide-resistant HP100 cells. Cancer Res. 2001 Aug. 1; 61(15):5885-94
36. Wang A S, Dreesen O. Biomarkers of Cellular Senescence and Skin Ageing. Front Genet. 2018 Aug. 23; 9: 247.
37. Watson R E, Griffiths C E, Craven N M, Shuttleworth C A, Kielty C M. Fibrillin-rich microfibrils are reduced in photoaged skin. Distribution at the dermal-epidermal junction. J Invest Dermatol. 1999 May; 112(5):782-7.
38. Feru J, Delobbe E, Ramont L, Brassart B, Terryn C, Dupont-Deshorgue A, Garbar C, Monboisse J C, Maquart F X, Brassart-Pasco S. Ageing decreases collagen IV expression in vivo in the dermo-epidermal junction and in vitro in dermal fibroblasts: possible involvement of TGF-β1. Eur J Dermatol. 2016 Aug. 1; 26(4):350-60.
39. Simmers P, Gishto A, Vyavahare N, Kothapalli C R. Nitric oxide stimulates matrix synthesis and deposition by adult human aortic smooth muscle cells within three-dimensional cocultures. Tissue Eng Part A. 2015 April; 21(7-8):1455-70.
40. Frank S, Kämpfer H, Wetzler C, Pfeilschifter J. Nitric oxide drives skin repair: novel functions of an established mediator. Kidney Int. 2002 March; 61(3):882-8.
41. Hummel S G, Fischer A J, Martin S M, Schafer F Q, Buettner G R. Nitric oxide as a cellular antioxidant: a little goes a long way. Free Radic Biol Med. 2006 Feb. 1; 40(3):501-6.

1. An injectable composition comprising:
non cross-linked sodium hyaluronate with a molecular weight between 100 and 400 kDa in a concentration between 7 and 20 mg/ml,
non cross-linked sodium hyaluronate with a molecular weight of at least 2000 kDa, in a concentration between 10 and 25 mg/ml,
a mixture of amino acids consisting of Glycine in a concentration between 6 and 12.5 mg/ml, L-Proline and/or L-Hydroxyproline in a concentration between 5 and 8 mg/ml, L-Alanine in a concentration between 1 and 5 mg/ml, L-Valine in a concentration between 1 and 5 mg/ml, L-Leucine in a concentration between 1 and 5 mg/ml, L-Lysine HCl in a concentration between 1 and 5 mg/ml, L-arginine HCl in a concentration between 1 and 5 mg/ml.

2. The composition according to claim 1, wherein the amino acid mixture consists of 9 mg/ml of Glycine, 6.5 mg/ml of L-Proline, 2 mg/ml of L-Alanine, 2.5 mg/ml of L-Valine, 1 mg/ml of L-Leucine, 2.5 mg/ml of L-Lysine HCl, 1.5 mg/ml of L-Arginine HCl.

3. The composition according to claim 1, wherein sodium hyaluronate with a molecular weight between 100 and 400 kDa is present in a concentration of 16 mg/ml and sodium hyaluronate with a molecular weight of at least 2000 kDa is present in a concentration of 16 mg/ml.

4. The composition according to claim 1, wherein sodium hyaluronate with a molecular weight between 100 and 400 kDa is present in a concentration of 12 mg/ml and sodium hyaluronate with a molecular weight of at least 2000 kDa is present in a concentration of 20 mg/ml.

5. The composition according to claim 1, wherein sodium hyaluronate is present in a total concentration greater than 25 mg/ml.

6. The composition according to claim 1, wherein said composition has a pH between 6.8 and 7.5.

7. The composition according to claim 1, comprising at least one of: physiological solution, pharmaceutically acceptable excipients or adjuvants, a phosphate buffer, or an anesthetic agent, in a concentration ranging from 0.1% to 0.4%.

8. The composition according to claim 1, further comprising at least one of
Acetyldecapeptide 3 of SEQ ID NO: 1, Oligopeptide 24 of SEQ ID NO: 2, Acetyltetrapeptide 5 of SEQ ID NO: 3, Vialox Pentapeptide-3 of SEQ ID NO: 4, Acetyl Hexapeptide 8 of SEQ ID NO: 5, Myristoyl Pentapeptide-8 of SEQ ID NO: 6, peptide GHK-Cu of sequence Gly-His-Lys-Cu, Tripeptide-29 of sequence H-Gly-Pro-Hyp-OH, Octapeptide-3 of SEQ ID NO: 7, MATRIXYL of SEQ ID NO: 8, or Hexapeptide of SEQ ID NO: 9,
each peptide at a final concentration between 0.005 mg/ml and 0.080 mg/ml.

9. A kit comprising an injectable composition according to claim 1, in the form of a gel, comprised in a pre-filled syringe and optionally comprising instructions for use.

10. The composition or kit according to claim 9, for use in the treatment of skin deterioration and/or senescence, elastosis and dermoepidermal atrophy caused by oxidative stress.

11. The use of the composition or kit according to claim 9 for cosmetic applications, for the treatment of at least one of: photoageing, cutaneous depressions, scars, imperfections and asymmetries of the face, wrinkles and lines of the skin, glabellar wrinkles, nasolabial folds, chin folds, marionette wrinkles, buccal wrinkles, peri-oral wrinkles, or crow's feet.

12. The use of the composition or kit according to claim 9 to stimulate collagen synthesis.

13. A non-therapeutic method of skin treatment of a subject, comprising the intradermal injection of the composition according to claim 1.

14. The composition according to claim 2, wherein sodium hyaluronate with a molecular weight between 100 and 400 kDa is present in a concentration of 12 mg/ml and sodium hyaluronate with a molecular weight of at least 2000 kDa is present in a concentration of 20 mg/ml.

15. The composition according to claim 2, wherein sodium hyaluronate is present in a total concentration greater than 25 mg/ml.


SEQUENCE LISTING (9 sequences)

SEQ ID NO: 1 - protein, 10 aa, synthetic construct
  MOD_RES 1: note = ACETYLATION
  MOD_RES 10: note = Tyr-NH2
YRSRKYTSWY

SEQ ID NO: 2 - protein, 13 aa, synthetic construct
RGDGCMYIEGGGG

SEQ ID NO: 3 - protein, 4 aa, synthetic construct
  MOD_RES 1: note = bAla
  MOD_RES 1: note = acetylation
AHSH

SEQ ID NO: 4 - protein, 5 aa, synthetic construct
  MOD_RES 5: note = alaninamide
GPRPA

SEQ ID NO: 5 - protein, 6 aa, synthetic construct
  MOD_RES 1: note = Acetylated
  MOD_RES 6: note = argininamide
EEMQRR

SEQ ID NO: 6 - protein, 5 aa, synthetic construct
  LIPID 1: note = MYRISTATE
  MOD_RES 5: note = lysinamide
RGDGK

SEQ ID NO: 7 - protein, 8 aa, synthetic construct
  MOD_RES 1: note = acetylated
  MOD_RES 8: note = Asparagine
EEMQRRAD

SEQ ID NO: 8 - protein, 5 aa, synthetic construct
KTTKS

SEQ ID NO: 9 - protein, 6 aa, synthetic construct
VGVAPG